(19) United States
(12) Patent Application Publication
(10) Pub. No.: US 2003/0149284 A1
(43) Pub. Date: Aug. 7, 2003

(54) Method of synthesizing a paclitaxel derivative

(76) Inventors: James Leslie Douglas, Montreal West (CA); Brian Francis Kaller, Candiac (CA); Lynda M. Moran, Mornington (IE)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/306,710

(22) Filed: Nov. 27, 2002

Related U.S. Application Data
(60) Provisional application No. 60/333,558, filed on Nov. 27, 2001.

(51) Int. Cl. C 07D 3 5/14

(52) U.S. Cl. 549/510

(57) ABSTRACT

A process for synthesizing paclitaxel derivative compounds useful for the treatment of cancer, comprises protecting the hydroxyl group at C-2′ position of a paclitaxel compound by reacting the same with a protecting group reagent to provide a protecting group (PG) at the C-2′ position, converting the hydroxyl group at C-7 position of the paclitaxel compound to a methylthiomethyl ether, and deprotecting the C-2′ position hydroxyl group through the removal of the protecting group reagent, thus yielding the final desired paclitaxel derivative product.

This application claims a benefit of priority from U.S. application Ser. No. 60/333,558, filed Nov. 27, 2001, the entire disclosure of which is herein incorporated by reference.

### FIELD OF THE INVENTION

The present invention relates generally to the synthesis of a paclitaxel derivative compound which is useful as an antitumor agent.

### BACKGROUND OF THE INVENTION

Paclitaxel (TAXOL®), a diterpene taxane compound, is a natural substance extracted from the bark of the Pacific yew tree, Taxus brevifolia. In studies, it has been shown to, possess excellent antitumor activity against a range of tumors in in vivo animal models.

Paclitaxel is a complex diterpenoid which comprises a bulky, fused ring system and an extended sidechain that is required for activity. The structure of paclitaxel is shown below along with the conventional numbering system for molecules belonging to the class of compounds, known as taxanes such numbering system is also used in this application.

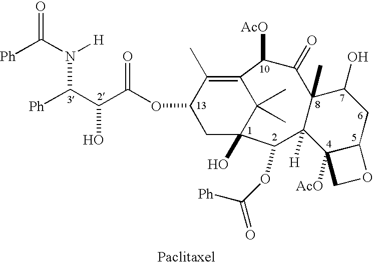

The particular carbon on the taxane structure shall be indicated throughout this application by a “C-position number”, which represents the carbon on the taxane according to the above numbering system. For example, “C-13” refers to the carbon at position 13 on the taxane ring as shown above, having the extended sidechain coupled thereto.

Derivatives of paclitaxel possess varying degrees of pharmacological activity. Investigations into the synthesis and evaluation of such paclitaxel derivative compounds have been made in an effort to develop safe, convenient, and efficacious drug formulations useful for the treatment of cancer in warm-blooded animals including humans. Since the discovery of paclitaxel, over one hundred compounds having a related structure have been isolated from various species of Taxus and/or made synthetically.

One paclitaxel derivative having desirable antitumor properties, is the compound, 7-O-methylthiomethyl paclitaxel (hereinafter referred as “7-O-MTM paclitaxel”) which differs structurally from paclitaxel at the C-7 position on the taxane ring. 7-O-MTM paclitaxel is a known antitumor agent currently under study in clinical trials. Studies involving 7-O-MTM paclitaxel have shown promising results in the treatment of gastrointestinal and colorectal cancers where paclitaxel has been found to be less effective.

It is known that 7-O-MTM paclitaxel may be produced by synthetic processes. However, such known processes have produced 7-O-MTM paclitaxel in yields or purity levels inadequate for efficient commercial production. For example, one known route for synthesizing the 7-O-MTM paclitaxel is described in the U.S. Pat. No. 5,646,176, the content of which is incorporated herein by reference. The synthesis described in the reference produces unwanted by-products, thus requiring chemical separation technology (e.g., flash chromatography on silica gel) to recover and purify the final product (i.e. 7-O-MTM paclitaxel). The use of chemical separation technology significantly diminishes the efficiency and cost effectiveness of the synthesis and reduces the likelihood that the synthesis can be effectively employed on a commercial scale. Another process is described in WO96/00724 in which triethylsilyl (TES) and trichoroethoxycarbonyl (TROC) are used as protecting group reagents to protect the 2′ position of a 7′-substituted taxol derivative.

Despite known methods for synthesizing 7-O-MTM paclitaxel, there remains a need for a process which produces a sufficiently pure product at desirable yields and which is especially suitable for commercial production.

### SUMMARY OF THE INVENTION

The present invention is directed to a new, useful and efficient process for the synthesis of 7-O-methylthiomethyl paclitaxel, or 7-O-MTM paclitaxel, which generally comprises protecting the hydroxyl group at the C-2′ position of paclitaxel with a protecting group reagent highly selective for the C-2′ hydroxyl group selected from trialkylsilyl halides and dialkylalkoxysilyl halides with the proviso that triethylsilyl chloride is excluded, converting the hydroxyl group at the C-7 position of the 2′-protected paclitaxel to a methylthiomethyl ether, and removing the protecting group reagent from the C-2′ position. The novel process provides a simple, efficient, and cost effective synthesis of 7-O-MTM-paclitaxel that is especially suitable for use in large-scale production.

One aspect of the present invention is directed to a process for synthesizing the paclitaxel derivative compound, 7-O-MTM paclitaxel of formula (1),

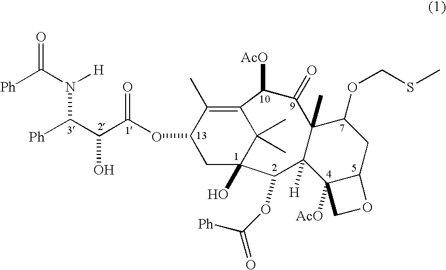

comprising the steps of:

(a) reacting paclitaxel with a protecting group (PG) reagent selected from the group consisting of trialkylsilyl halides and dialkylalkoxysilyl halides with the proviso that triethylsilyl chloride is excluded, to provide a C-2′ protected paclitaxel of formula (2) having a protecting group (PG) in the C-2′ position;

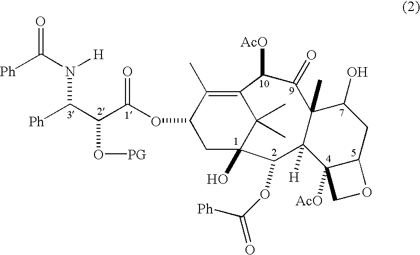

(b) reacting the C-2′ protected paclitaxel of formula (2) with a methylthiomethylation reagent to form a C-2′ protected, 7-O-methylthiomethyl paclitaxel of formula (3); and

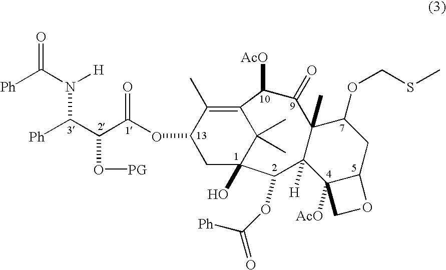

(c) removing the protecting group (PG) to form the 7-O-methylthiomethyl paclitaxel of formula (1).

### DETAILED DESCRIPTION OF THE INVENTION

The present invention is generally directed to a process for synthesizing a 7-O-MTM paclitaxel at relatively high yields and purity at reduced costs suitable for large-scale commercial production. The present invention encompasses a novel method by which paclitaxel is converted into a C-2′ protected paclitaxel by the reaction between the hydroxyl group at the C-2′ position and a protecting group reagent having a high affinity for the C-2′ hydroxyl group. The resulting C-2′ protected paclitaxel having a protecting group (PG) in the C-2′ position is treated with a methylthiomethylation reagent (e.g. dimethylsulfide) whereby the hydroxyl group at the C-7 position is converted into a methylthiomethyl ether. The C-2′ protected, 7-O-MTM paclitaxel is then converted into 7-O-MTM paclitaxel by the removal of the C-2′ protecting group. The resulting compound exhibits desirable antitumor properties.

The term “protecting group reagent” defined herein is a trialkylsilyl halide or a dialkylalkoxysilyl halide with the proviso that triethylsilyl chloride is excluded, which reacts with a hydroxyl group (i.e. at the C-2′ position of paclitaxel), binds chemically to the remaining oxygen radical, and stays bound as a protecting group (PG) through reactions where the methylthiomethyl group is chemically bound to the oxygen. The protecting group is thereafter removed (either chemically, in vitro, or in vivo) from the oxygen radical by known methods to restore the hydroxyl group. A preferred protecting group reagent is t-butyldimethylsilyl chloride.

“Alkyl” means a straight or branched saturated carbon chain having one or more carbon atoms; examples include methyl, ethyl (excluding triethylsilyl), n-propyl, isopropyl, n-butyl, sec-butyl, isobutyl, t-butyl, n-pentyl, sec-pentyl, isopentyl, and n-hexyl. “Alkoxy” means a straight or branched saturated carbon chain having one or more carbon atoms attached with oxygen; examples include methoxy, ethoxy, n-propoxy, isopropoxy, n-butoxy, sec-butoxy, isobutoxy, t-butoxy, n-pentoxy, sec-pentoxy, isopentoxy, and n-hexoxy. “Methylthiomethyl” (also abbreviated as MTM) generically refers to the group —CH₂SCH₃.

The term “methylthiomethylation” as used herein refers to a reaction which results in the addition of a methylthiomethyl ether group to the C-7 position of paclitaxel. The term “methylthiomethylation reagent” refers to any compound which is capable of initiating the methylthiomethylation reaction and provides a methylthiomethyl ether group at the C-7 position.

The present invention is broadly directed to a process for the efficient synthesis of 7-O-MTM paclitaxel. In the process of the present invention, paclitaxel is treated with a protecting group reagent as defined herein having a high affinity for the hydroxyl group at the C-2′ position of paclitaxel. The preferred trialkylsilyl halide protecting group reagent is t-butyldimethylsilyl chloride, and the preferred dialkylalkoxysilyl halides include dimethylmethoxysilyl chloride, diethylmethoxysilyl chloride, and diisopropylmethoxysilyl chloride. A particularly preferred dialkylalkoxysilyl halide protecting group reagent is dimethylmethoxysilyl chloride.

The resulting C-2′ protected paclitaxel is treated with a methylthiomethylation reagent whereby the hydroxyl group at the C-7 position is preferentially converted to a methylthiomethyl ether. In the final step of the reaction sequence, the C-2′ protected paclitaxel is deprotected through the removal of the protecting group (PG) at the C-2′ position to restore the 2′-hydroxyl group. The deprotecting procedure may be accomplished through conventional methods well known in the art such as acid- or base-catalyzed hydrolysis, hydrogenolysis, reduction, and the like. Deprotecting methodologies may be found in standard texts such as Greene and Wutz, Protective Groups in Organic Synthesis, 2d Ed., John Wiley & Sons, 1991; and McOmie, Protective Groups in Organic Chemistry, Plenum Press, 1975, incorporated herein by reference.

General Synthesis Procedures:

The 7-O-MTM paclitaxel compound employed in this invention may be prepared from readily available starting materials using the following general methods and procedures. It will be understood that where typical or preferred process conditions (i.e. reaction temperatures, times, mole ratios of reactants, solvents, pressures, etc.) are given, other process conditions may also be used unless otherwise stated. Optimum reaction conditions may vary with the particular reactants or solvents used, however such reaction conditions may be determined by one of ordinary skill in the art through routine optimization procedures.

The abbreviations used herein are conventional abbreviations widely employed in the art. Some of which are:

| | |
|---|---|
| Ac | Acetyl |
| BPO | Benzoyl peroxide |
| CyH | Cyclohexane |
| DCP | Dicyclohexyl phthalate |
| DBU | 1,8-Diazabicyclo[5.4.0]undec-7-ene |
| DI H₂O | Deionized water |
| DMF | N,N-Dimethylformamide |
| DMS | Dimethylsulfide |
| EtOAc | Ethyl acetate |
| h | Hour(s) |
| HCl | Hydrochloric acid |
| H₂O | Water |
| IPA | Isopropyl alcohol |
| min | Minute(s) |
| MTBE | Methyl-t-butyl ether |
| NaHCO₃ | Sodium bicarbonate |
| NaOH | Sodium hydroxide |
| Ph | Phenyl |
| TBDMS-Cl | t-Butyldimethylsilyl chloride |
| 3 HF.TEA | Triethylamine trihydrofluoride |

A. Production of C-2′ Protected-Paclitaxel

The process for synthesizing C-2′ protected paclitaxel is illustrated in Reaction Scheme 1.

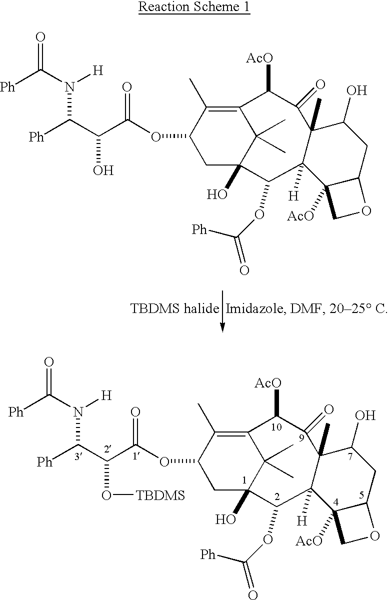

As illustrated in Reaction Scheme 1, paclitaxel is treated with a protecting group reagent as defined herein having a high affinity for the hydroxyl group at the C-2′ position, and a base such as imidazole, triethylamine, diisopropylethylamine, 4-dimethylaminopyridine, or DBU, preferably imidazole. A preferred trialkylsilyl halide protecting group reagent is t-butyldimethylsilyl chloride, while a preferred dialkylalkoxysilyl halide is dimethylmethoxysilyl chloride. The reaction is carried out in an inert organic solvent such as N,N-dimethylformamide (DMF), N,N-dimethylacetamide, N-methylpyrrolidone or the like, preferably DMF, at a temperature conducive to product formation; typically the reaction is carried out at a temperature in the range of from about 20°to 25° C. Imidazole is used in excess relative to paclitaxel, preferably in the range of from about 2.5 to 2.8 equivalents to one equivalent of paclitaxel.

According to Reaction Scheme 2 shown below, the C-2′ protected paclitaxel as prepared in Reaction Scheme 1 above, is reacted with a methylthiomethylation reagent to replace hydrogen of the hydroxyl group at the C-7 position with a methylthiomethyl group. The resulting product is then deprotected to remove the protecting group from the C-2′ position to thereby form 7-O-MTM-paclitaxel.

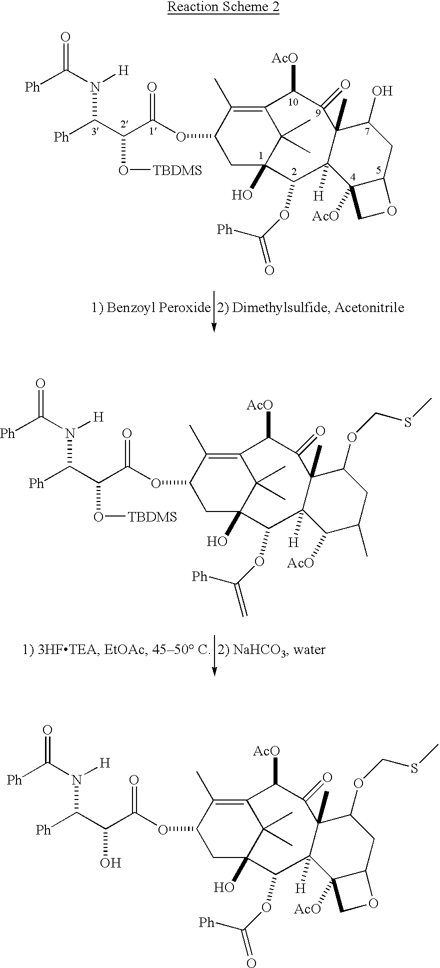

The addition of a protecting group reagent in accordance with the present invention is advantageously provided by t-butyldimethylsilyl chloride (TBDMS-Cl), which results in the formation of the corresponding 2′-TBDMS-protected paclitaxel. It is noted that the present invention may include the use of other protecting group reagents for forming protecting groups as defined herein of similar high affinity for the C-2′ position hydroxyl group.

In a preferred form of the present invention, each equivalent of paclitaxel is reacted with at least one equivalent, preferably from about 2 to 3 lo equivalents of TBDMS-Cl, and more preferably about 2.2 equivalents. The equivalent excess amount of TBDMS-Cl ensures complete protection of the hydroxyl group at the C-2′ position of paclitaxel. The reaction is preferably conducted at a temperature of from about 20° C. to 25° C. It is noted that the equivalents of TBDMS-Cl and imidazole may be adjusted to compensate for is the moisture content in the paclitaxel starting material. For every equivalent of H₂O present in the paclitaxel starting material, about one equivalent each of TBDMS-Cl and imidazole is used to compensate for the additional moisture content. ′ Upon completion of the TBDMS protection reaction, the C-2′ protected paclitaxel is recovered through conventional product recovery methods including precipitation, filtration, distillation, and the like.

B. Production of C-2′ Protected, 7-O-MTM Paclitaxel and Synthesis of 7-O-MTM Paclitaxel Therefrom ′ The C-2′ protected paclitaxel is reacted with a methylthiomethylation reagent such as dimethylsulfide (DMS) in the presence of an organic peroxide such as benzoyl peroxide. The reaction is carried out in an inert organic solvent such as acetonitrile, methylene chloride and the like at a temperature conducive to product formation; typically the reaction is carried out at a temperature range of from about −40° C. to about ambient temperature. Dimethylsulfide and benzoyl peroxide are preferably used in excess relative to the amount of C-2′ protected paclitaxel, and dimethylsulfide is preferably used in excess relative to the amount of benzoyl peroxide.

In a preferred form of the present invention, dimethylsulfide is used in the amount ranging from about 8 to 12 equivalents, more preferably 10 equivalents; and benzoyl peroxide is used in the amount ranging from about 3 to 5 equivalents, more preferably 4 equivalents. Preferably, the reaction is carried out in acetonitrile at a temperature ranging from about −10° to 20° C., more preferably from about −5° to 0° C. For safety purposes, the benzoyl peroxide is preferably present in the form of a mixture of benzoyl peroxide and a phthalate ester such as cyclohexyl phthalate. Any remaining unreacted benzoyl peroxide may be quenched by treatment with a base such as sodium hydroxide after completion of the methylthiomethylation reaction.

The methylthiomethylation reaction yields the corresponding C-2′ protected, 7-O-methylthiomethyl compound. The C-2′ protected, 7-O-MTM-paclitaxel is subsequently treated with a deprotecting reagent to remove the C-2′ protecting group to form the final product, 7-O-MTM paclitaxel. The deprotecting reagents suitable for removing the protecting groups employed in the present invention include such compounds as triethylamine trihydrofluoride or trifluoroacetic acid.

In a preferred form of the present invention, the C-2′ protected, 7-O-MTM paclitaxel is treated with triethylamine trihydrofluoride in an organic solvent such as ethyl acetate (EtOAc). The amount of triethylamine trihydrofluoride preferably ranges from about 1.5 to 2.0 equivalents, more preferably about 1.7 equivalents. Alternatively, the reaction may use trifluoroacetic acid as an acceptable substitute for triethylamine trihydrofluoride. Once the TBDMS protecting group is removed from the C-2′ position, the final product, 7-O-MTM paclitaxel, may then be recovered through conventional product recovery methods including precipitation, filtration, distillation, and the like.

The foregoing discussion discloses and describes merely exemplary embodiments of the present invention. One skilled in the art will readily recognize from such discussion, and from the examples that follow, and from the claims, that various changes, modifications, and variations can be made therein without departing from the spirit and scope of the invention.

It is further noted that one of ordinary skill in the art can, using the above description, perform the processes disclosed and prepare the full scope of the intermediates and compounds of the present invention. The following examples further exemplify the general procedure for the preparation procedures inherent in the synthesis of 7-O-MTM paclitaxel from paclitaxel. The following examples are submitted for illustrative purposes only and are not intended to limit the invention as encompassed by the claims forming part of the application.

### EXAMPLE 1

Synthesis of C-2′ Protected Paclitaxel from Paclitaxel

The following procedure was used for producing an intermediate product, C-2′ protected paclitaxel.

A vessel equipped with a mechanical agitator, a thermocouple probe and nitrogen gas inlet was flushed with nitrogen gas. The vessel was then charged with paclitaxel (20.00 g, 23.4 mmol) and N,N-dimethylformamide (DMF) (30 to 40 mL) and then flushed with nitrogen gas. The resulting paclitaxel slurry was agitated for 10 to 20 min at a temperature of from about 20° to 25° C. Imidazole (4.49 g, 2.82 eq.) was added to the paclitaxel slurry along with DMF (2.5 mL) as a rinse. The paclitaxel slurry was then agitated for 10 min to yield a clear solution.

t-Butyldimethylsilyl chloride as a protecting group reagent (TBDMS-Cl) (7.84 g, 2.22 eq.) was added to the clear solution along with DMF (˜2.5 mL) as a rinse. The resulting solution was agitated for about 6 h. Methyl t-butyl ether (MTBE) (200 mL) was added to the reaction mixture to form a milky mixture. The milky mixture was washed with dilute hydrochloric acid and water. The washed organic layer was concentrated. Cyclohexane (CyH) was added to the concentrated organic layer resulting in crystallization. The slurry was concentrated and cooled to a temperature of from about 20° to 25° C.

The first intermediate product, C-2′ protected paclitaxel having t-butyldimethylsilyl as a protecting group, was then collected by vacuum filtration. The resulting filter cake was washed with CyH (60 mL) and dried in a vacuum to yield 22.24 g of the first intermediate product, C-2′ protected paclitaxel.

### Example 2

General Procedure for Coupling Dimethylsulfide to C-2′ Protected Paclitaxel

The following procedure was used to produce the intermediate product C-2′ protected, 7-O-MTM paclitaxel. A methylthiomethylation reaction was carried out on the first intermediate product (C-2′ protected paclitaxel) produced in accordance with Example 1 to yield a second intermediate product, C-2′ protected, 7-O-MTM paclitaxel.

C-2′ protected paclitaxel (20.0 g, 20.7 mmol), a mixture of benzoyl peroxide (BPO) and dicyclohexyl phthalate (DCP) (40.0 g, containing 20.0 g, 82.6 mmol, 4.0 eq. of BPO), were charged to a reactor vessel which was equipped with an agitator, thermocouple, a cooling and heating system and gas inlet and out. Oxygen was removed from the reactor vessel and replaced with inert nitrogen gas for safety purposes. Acetonitrile (160 g, 204 mL) was added to the mixture in the vessel to form a slurry. The slurry was agitated and cooled to about 0° C. Upon cooling, dimethylsulfide (DMS) (12.9 g, 207 mmol, 10.0 eq.) was added to the slurry while maintaining the temperature until completion of the methylthiomethylation reaction. A high performance liquid chromatography (HPLC) apparatus was used to determine completion of the reaction.

A 1N NaOH solution at a temperature of from about 0° to 20° C. was added to the reaction mixture. MTBE (80.0 g, 108 mL) was added to the reaction mixture. The reaction mixture was then raised to a temperature of about 20° C. and maintained at the temperature for about 1 h. The organic phase was separated, washed two times with water, and then concentrated by solvent distillation to a volume of about 60 mL. Isopropyl alcohol (IPA) was added and concentration by distillation continued until all MTBE and acetonitrile were replaced by IPA. IPA was further added to give a total volume of about 455 mL. About 40 mL of ethyl acetate (EtOAc) was added to the mixture. The resulting mixture was heated to a temperature of from about 70° to 80° C., until complete dissolution. The mixture was then cooled to about 20° C. to yield a crystalline product. The crystalline product was collected by filtration and dried to yield 19.1 g of the second intermediate product, C-2′ protected, 7-O-MTM paclitaxel.

### Example 3

Synthesis of 7-O-MTM Paclitaxel from C-2′ Protected, 7-O-MTM Paclitaxel

The following procedure was used for producing the final product, 7-O-MTM paclitaxel. The protecting group, TBDMS, was removed from the second intermediate product (C-2′ protected, 7-O-MTM paclitaxel) produced in accordance with Example 2, to yield a final product, 7-O-MTM paclitaxel.

C-2′ protected, 7-O-MTM paclitaxel (20.0 g, 19.4 mmol) and EtOAc (100 mL) were added to a 500 mL 3-necked flask which was equipped with a mechanical agitator, a thermocouple probe, and gas inlet and outlet. Triethylamine trihydrofluoride (3HF.TEA) (5.4 mL, 33.1 mmol, 1.70 eq.) was added to the reaction mixture under a nitrogen gas atmosphere at a temperature of from about 20° to 25° C. The reaction mixture was heated to a temperature of from about 45° to 50° C. and agitated for about at least 4 to 5 h. A cloudy solution resulted. The cloudy solution was cooled to a temperature of from about 20° to 25° C. EtOAc (140 mL) was added to the cloudy solution. The organic solution was then washed with sodium bicarbonate (NaHCO₃) solution (60 mL, 8% w/w). The organic layer was washed with about two 70 mL portions of deionized water (DI H₂O). The organic layer was then polish filtered. The filtered organic layer was concentrated through several cycles with addition of isopropyl alcohol (IPA) so as to remove all residual EtOAc.

IPA was added to the organic layer to reach a volume of about 20 mL/(g input). The organic layer was then seeded with 7-O-MTM paclitaxel. The organic layer was then cooled to a temperature of about 20° to 25° C. for over at least about 1 h and maintained at that temperature for at least about 4 h. The organic layer was filtered to collect the final product in the form of a filter cake. The filter caked was washed with IPA and water. The slurry was then filtered and a filter cake of 7-O-MTM paclitaxel was collected. The filter cake was washed with DI H₂O. The wet cake was deliquored and dried at a temperature of from about 55° to 60° C. under reduced pressure until the level of residual IPA is less than 1%, thereby yielding 14.7 grams of the final free-flowing powder product. The corrected yield was 82 M% after crystallization from IPA.

What is claimed is:

1. A process for synthesizing a 7-O-methylthiomethyl paclitaxel of formula (1) 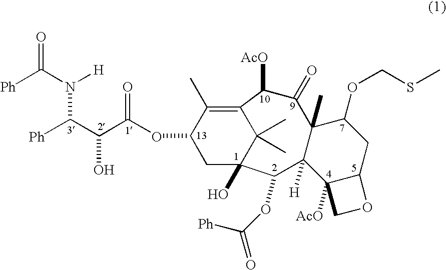
comprising the steps of:
(a) reacting paclitaxel with a protecting group reagent selected from the group consisting of trialkylsilyl halides and dialkylalkoxysilyl halides with the proviso that triethylsilyl chloride is excluded, to provide a C-2′ protected paclitaxel of formula (2) having a protecting group, PG, in the C-2′ position; 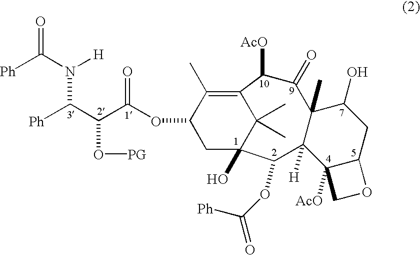
(b) reacting the C-2′ protected paclitaxel of formula (2) with a methylthiomethylation reagent to form a C-2′ protected, 7-O-methylthiomethyl paclitaxel of formula (3); and 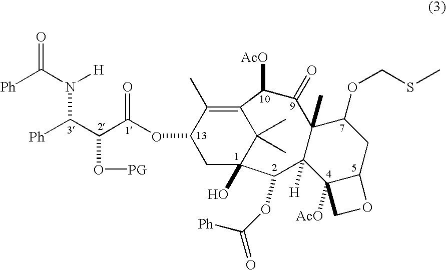
(c) removing the protecting group PG to form the 7-O-methylthiomethyl paclitaxel of formula (1).

2. The process of claim 1 wherein the methylthiomethylation reagent is dimethylsulfide.

3. The process of claim 1 wherein the trialkylsilyl halide protecting group reagent is t-butyldimethylsilyl chloride.

4. The process of claim 1 wherein the dialkylalkoxysilyl halide is selected from the group consisting of dimethylmethoxysilyl chloride, diethylmethoxysilyl chloride, and diisopropylmethoxysilyl chloride.

5. The process of claim 1 further comprising conducting the reaction of step (a) in the presence of an inert organic solvent and a base.

6. The process of claim 5 wherein:
the base is selected from the group consisting of imidazole, triethylamine, diisopropylethylamine, 4-dimethylaminopyridine, and DBU; and
the solvent is selected from the group consisting of N,N-dimethylformamide, N,N-dimethylacetamide, and N-methylpyrrolidone.

7. The process of claim 5 further comprising conducting the reaction of step (a) at a temperature in the range of from about 20° to 25° C.

8. The process of claim 1 further comprising conducting the reaction of step (b) in the presence of benzoyl peroxide and acetonitrile.

9. The process of claim 8 further comprising conducting the reaction of step (b) at a temperature in the range from about −40° C. to ambient temperature.

10. The process of claim 9 comprising conducting the reaction of step (b) at a temperature in the range of from about −10° to 20° C.

11. The process of claim 8 comprising conducting the reaction of step (b) in the presence of a phthalate ester.

12. The process of claim 11 wherein the phthalate ester is dicyclohexyl phthalate.

13. The process of claim 11 wherein the benzoyl peroxide and phthalate ester are present in equal molar ratio amounts.

14. The process of claim 1 wherein step (b) further comprises the steps of:
adding an organic solvent to the C-2′ protected 7-O-methylthiomethyl paclitaxel of formula (3); and
reacting the C-2′ protected 7-O-methylthiomethyl paclitaxel of formula (3) with a deprotecting reagent.

15. The process of claim 14 wherein the deprotecting reagent is selected from the group consisting of triethylamine trihydrofluoride and trifluoroacetic acid.

16. The process of claim 8 wherein the protecting group reagent used in the reaction of step (a) is a dialkylalkoxysilyl halide selected from the group consisting of dimethylmethoxysilyl chloride, diethylmethoxysilyl chloride and diisopropylmethoxysilyl chloride.
